(19) United States
(12) Patent Application Publication
(10) Pub. No.: US 2007/0166402 A1
(43) Pub. Date: Jul. 19, 2007

(54) Compositions, methods and kits for removing debris from an ocular area

(76) Inventors: Mitchell H. Friedlaender, La Jolla, CA (US); Harun Takruri, Newport Beach, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/637,136

(22) Filed: Dec. 12, 2006

Related U.S. Application Data
(60) Provisional application No. 60/749,079, filed on Dec. 12, 2005.

(51) Int. Cl. A61K 33/22; A61K 31/4172

(52) U.S. Cl. 424/659; 514/400

(57) ABSTRACT

The present invention is directed to compositions and kits for removing debris from an ocular area. The present invention is also directed to a method of removing debris from an ocular area.

This application claims the benefit of the filing date of U.S. Application No. 60/749,079, filed Dec. 12, 2005, which is incorporated by reference herein in its entirety.

### BACKGROUND OF THE INVENTION

1. Field of the Invention

The present invention is directed to compositions, methods and kits for removing debris from an ocular area.

2. Background Art

Eyelid pathology is one of the most common, yet one of the least treated medical conditions of the eye. Oil, dandruff, bacteria and environmental debris accumulate on the eyelids throughout the day. Additionally, medicinal gels, ointments, suspensions and solutions may be added to the eyelids. Women are likely to add eye makeup to the material that accumulates naturally on the lids and lashes. Additionally, pathological conditions such as eye infections and blepharitis may result in deposits in the form of mucin encrustations or dead tissue scales. Cleaning of the eyelid and surrounding area varies in effectiveness, even with commercially available eye makeup removers.

Eyelid debris often results in chronic irritation, or inflammation of the eye. This condition is termed “blepharitis.” In both men and women, blepharitis can lead to redness of the lid margins and conjunctiva (the white part of the eye). In fact, the most common cause of “red eyes” is blepharitis. Red eyes, caused by blepharitis, account to some extent for the common usage of vasoconstrictor eye drops. However, these vasoconstrictor eye drops do not address one of the roots of the problem, the problem being debris that accumulates on the eyelids, and the low grade staphylococcal infections that ensue.

Several commercial products for cleaning eyelids currently utilize synthetic detergents and cleansers to facilitate cleaning. Such synthetic detergents and cleansers often cause discomfort and irritation if they contact the sensitive tissues of the eye during application. Additionally, they generally have an undesired “soapy” feel that results in regimen compliance problems. For example, one commercial eyelid cleanser contains, among other ingredients, glyceryl tallowate, disodium laureth sulfosuccinate, cocoamido propyl amine oxide, and PEG-80 glyceryl cocoate. Another commercial eye makeup remover contains, among other ingredients, poloxamer 184, disodium lauroamphodiacetate and sodium trideceth sulfate. A need exists in the art for products that are capable of cleaning an ocular area without causing discomfort and irritation.

### BRIEF SUMMARY OF THE INVENTION

The present invention is directed to a composition for removing debris from an ocular area comprising: (a) about 0.1% to about 5.0% w/v boric acid; (b) about 0.001% to about 1.0% w/v edetate disodium; and (c) about 0.001% to about 1.0% w/v benzalkonium chloride.

In some embodiments, the removed debris is a cosmetic product. The cosmetic product can include mascara, eye shadow makeup, eyeliner (creams or pencils), moisturizer, makeup base, concealer, eyebrow pencil, glitter and combinations thereof. Alternatively, the removed debris can be a biological product resulting from an eye infection or eye inflammation, e.g., encrusted mucin or dead tissue scales.

In some embodiments, the composition further comprises an active agent. In some embodiments, the active agent is selected from the group consisting of, but not limited to, an antihistamine, vasoconstrictor, mast cell stabilizer, nonsteroidal anti-inflammatory agent, and combinations thereof.

In some embodiments, the active agent is an antihistamine. In some embodiments, the antihistamine is selected from the group consisting of antazoline phosphate, pheniramine maleate, and combinations thereof.

In some embodiments, the active agent is a vasoconstrictor. In some embodiments, the vasoconstrictor is selected from naphazoline hydrochloride, tetrahydrozoline hydrochloride or oxymetazoline hydrochloride. In some embodiments, wherein the vasoconstrictor is about 0.001% to about 0.1% w/v of the composition.

In some embodiments, the active agent is a mast cell stabilizer. In some embodiments, the mast cell stabilizer is selected from the group consisting of olopatadine, ketotifen, epinastine, azelastine, pemirolast, nedocromil, and combinations thereof.

In some embodiments, the active agent is a nonsteroidal anti-inflammatory agent. In some embodiments, the nonsteroidal anti-inflammatory agent is selected from the group consisting of ketorolac tromethamine, flurbiprofen, diclofenac, nepafenac, and combinations thereof.

In some embodiments, the composition of the present invention further comprises an artificial tear solution. In some embodiments, the artificial tear solution comprises water, a cellulose derivative, and an antimicrobial agent.

Examples of a vasoconstrictor include naphazoline hydrochloride, tetrahydrozoline hydrochloride or oxymetazoline hydrochloride. The vasoconstrictor can be about 0.001% to about 0.1% w/v of the composition.

The composition of the present invention can further comprise a UV-absorbing compound. Examples of UV-absorbing compounds include vitamin E acetate, vitamin E succinate, and vitamin E polyethylene glycol succinate. The UV-absorbing compound can be about 0.1% to about 10% w/v of the composition.

In some embodiments, the composition further comprises an additional ingredient selected from the group consisting of a buffer, tonicity adjuster, viscosity modifier, performance enhancer, and combinations thereof. For example, the ingredient can be selected from sodium chloride, sodium phosphates, potassium phosphates, citric acid, sodium citrate, acetic acid, sodium acetate, glycerin, propylene glycol, polyethylene glycol, and polysorbates.

The ocular area as described herein can include the eyelid (upper and/or lower), the margin of the eyelid, the eyelashes, the eye brow, or combinations thereof.

The concentration of the boric acid, edetate disodium, and benzalkonium chloride can vary. In some instances, the composition comprises about 0.5% to about 3% w/v boric acid. In some instances, the composition comprises about 0.01% to about 0.4% w/v edetate disodium. In some instances, the composition comprises about 0.005% to about 0.04% w/v benzalkonium chloride.

In some embodiments, the composition for removing debris from an ocular area comprises: (a) 1% w/v boric acid; (b) 0.2% w/v edetate disodium; (c) 0.01% w/v benzalkonium chloride; (d) 0.005 molar phosphate buffer; (e) 0.4% w/v sodium chloride; (f) sodium hydroxide/HCl sufficient to adjust the pH to 7.8; and (g) purified water. In another embodiment, the composition for removing debris from an ocular area comprises: (a) 1.5% w/v boric acid; (b) 0.2% w/v edetate disodium; and (c) 0.02% w/v benzalkonium chloride; (d) 0.005 molar phosphate buffer; (e) 0.007% w/v sodium chloride; (f) 0.6% w/v sodium borate decahydrate; (g) 0.01% w/v tetrahydrozoline hydrochloride; (h) sodium hydroxide/HCl sufficient to adjust the pH to 7.8; and (i) purified water. In yet another embodiment, the composition for removing debris from an ocular area comprises: (a) 1.5% w/v boric acid; (b) 0.2% w/v edetate disodium; and (c) 0.02% w/v benzalkonium chloride; (d) 0.02% w/v vitamin E acetate; (e) 2% w/v vitamin E polyethylene glycol succinate; (f) 0.6% w/v sodium borate decahydrate; (g) sodium hydroxide/HCl sufficient to adjust the pH to 7.8; and (h) purified water.

The present invention is also directed to a method for removing debris from an ocular area, the method comprising applying a wetted towelette to an ocular area to remove debris, the wetted towelette comprising a composition comprising: (i) about 0.1% to about 5.0% w/v boric acid; (ii) about 0.001% to about 1.0% w/v edetate disodium; and (iii) about 0.001% to about 1.0% w/v benzalkonium chloride.

In some embodiments, the invention is directed to a method for removing a cosmetic product from an ocular area, the method comprising applying a wetted towelette to an ocular area to remove the cosmetic product, the wetted towelette comprising a composition comprising: i) about 0.1% to about 5.0% w/v boric acid; ii) about 0.001% to about 1.0% w/v edetate disodium; and iii) about 0.001% to about 1.0% w/v benzalkonium chloride.

In some embodiments, the towelette is wetted immediately before the application to the ocular area. Alternatively, the towelette can be wetted substantially before the application to the ocular area. In some embodiments, the towelette is disposable.

In some embodiments, the present invention is directed to a method for treating one of more conditions selected from blepharitis, dry eyes, red eyes, and allergic conjunctivitis, the method comprising: (a) applying a composition to an ocular area containing debris, the composition comprising (i) about 0.1% to about 5.0% w/v boric acid; (ii) about 0.001% to about 1.0% w/v edetate disodium; and (iii) about 0.001% to about 1.0% w/v benzalkonium chloride; and (b) removing the debris from the ocular area. In some embodiments, the composition further comprises a vasoconstrictor, antihistamine, mast cell stabilizer, and/or nonsteroidal anti-inflammatory. In some embodiments, the applying is performed by contacting a towelette comprising the composition to the ocular area.

The present invention is also directed to a kit for removing debris from the ocular area, the kit comprising: (a) a composition comprising: (i) about 0.1% to about 5.0% w/v boric acid; (ii) about 0.001% to about 1.0% w/v edetate disodium; and (iii) about 0.001% to about 1.0% w/v benzalkonium chloride; and (b) one or more towelettes. In some instances, the kit of the present invention further comprises instructions for using the composition of (a) and the towelette of (b) to remove debris.

### DETAILED DESCRIPTION OF THE INVENTION

The invention provides a composition for removing debris from an ocular area comprising: (a) about 0.1% to about 5.0% w/v boric acid; (b) about 0.001% to about 1.0% w/v edetate disodium; and (c) about 0.001% to about 1.0% w/v benzalkonium chloride.

The present invention further provides a method for removing debris from an ocular area, the method comprising applying a wetted towelette to an ocular area to remove debris, the wetted towelette comprising a composition comprising: (i) about 0.1% to about 5.0% w/v boric acid; (ii) about 0.001% to about 1.0% w/v edetate disodium; and (iii) about 0.001% to about 1.0% w/v benzalkonium chloride.

In some embodiments, the invention provides for a kit for removing debris from the ocular area, the kit comprising (a) a composition comprising: (i) about 0.1% to about 5.0% w/v boric acid; (ii) about 0.001% to about 1.0% w/v edetate disodium; and (iii) about 0.001% to about 1.0% w/v benzalkonium chloride; and (b) one or more towelettes.

In the present invention, the towelette is wetted prior to application to the ocular area. In some embodiments, the towelette is wetted immediately before the application to the ocular area. Alternatively, the towelette can be wetted substantially before the application to the ocular area, e.g., the towelette can be wetted prior to the sale to a consumer. For example, the towelette of the present invention can be wetted by a manufacturer or distributor of a product, or alternatively by a pharmacist. The term “wetted” refers to the placement of the composition of the invention in contact with the towelette, and the ensuing permeation of the composition into the towelette.

The term “towelette” as used herein refers to any object suitable for applying the composition of the present invention to an ocular area. In some embodiments, the towelette suitably absorbs the composition of the present invention for convenient administration to an ocular area. Alternatively, in some embodiments the towelette does not absorb the composition of the present inventions. In some embodiments, the towelette has a surface textured to facilitate removal of debris from an ocular area. For example, in some embodiments the towelette can have a rough surface, bristled surface, or other raised surfaces which facilitate removal of debris. The towelette of the present invention can be constructed of various materials. For example, the towelette can be constructed of any cloth, e.g., terry cloth or other loop-weaved fabrics or textured fabrics, other cotton based products, e.g., cotton swabs or cotton balls or cotton gauze, non-cotton based fabrics, or paper-based products. Additionally, the towelette can include sheets of fabric, pads, swabs, and the like. In some embodiments, the towelette is disposable. Alternatively, the towelette can be washable and reused. The towelette used in the present invention can vary in size. For example, the towelette can be from ½ inch² to 12 inches² in size, or greater, preferably ½ inch² to 6 inches², more preferably ½ inch² to 3 inches², or 1 inch² to 2 inches².

The debris removed from the ocular area can vary. In some instances, debris can be a cosmetic product. The term “cosmetic product” as used herein refers to any compound or composition placed in the ocular area. “Cosmetic product” refers not only to compounds that directly alter the appearance of the ocular area (e.g., mascara), but also to compounds that can indirectly alter the ocular area (e.g., invisible moisturizers that moisturize the skin). Thus, the term “cosmetic products” should not be limited to products visible when applied to an ocular area. Cosmetic products can include, but are not limited to, mascara, eye shadow makeup, eyeliner (creams or pencils), moisturizer and makeup base (makeup foundation), concealer, eyebrow pencil, glitter and combinations thereof. In some embodiments, the cosmetic product can be an artificial eyelash, or an adhesive for an artificial eyelash. In some embodiments, the cosmetic product is waterproof or water resistant. In alternative embodiments, the cosmetic product is water soluble or water removable.

Alternatively, the debris removed can be a biological product, e.g., eyelashes, oil or dandruff. In some embodiments, the biological product results from an eye infection or eye-inflammation, e.g., blepharitis. The biological debris resulting from the eye-infection or eye-inflammation can be, but is not limited to, encrusted mucin or dead tissue scales. In some instances, the debris can be a foreign object, e.g., dust or other environmental debris.

The composition of the present invention comprises boric acid, edetate disodium, and benzalkonium chloride. In some embodiments, the composition further comprises an active agent. The term “active agent”, as used herein refers to a chemical compound, macromolecule, or composition of matter which, when administered to an organism (human or animal subject), induces a desired pharmacologic and/or physiologic effect by local and/or systemic action. The term therefore encompasses those compounds or chemicals traditionally regarded as drugs or biopharmaceuticals (including molecules such as peptides, proteins, nucleic acids). Examples of active agents include antibiotics and antiviral agents; analgesics and analgesic combinations, antiseptics, antihistamines; anti-inflammatory agents, hormones or steroids, vasodilators; proteins, peptides, and fragments thereof (whether naturally occurring, chemically synthesized or recombinantly produced); and nucleic acid molecules. In some embodiments, the active agent can be selected from the group consisting of, but not limited to, an antihistamine, vasoconstrictor, mast cell stabilizer, nonsteroidal anti-inflammatory agent, and combinations thereof. In some embodiments, the composition does not comprise an active agent, e.g., the composition does not comprise a vasoconstrictor, antihistamine, mast cell stabilizer, nonsteroidal anti-inflammatory, etc.

In some embodiments of the present invention, the active agent is a compound, macromolecule, or composition of matter, wherein the dosage amount required to treat a condition is not critical. For example, in embodiments wherein a towelette is used when applying the composition, then an inconsistent amount of the active agent can be applied to the ocular surface since varying amounts of the active agent may remain in the towelette. Additionally, since the size of an ocular surface can vary, and the amount of the composition which adheres or remains with the ocular area can vary, then it can be difficult to provide a consistent dosage amount. By way of example, if the composition comprising an active agent is applied to treat a red eye to an ocular area wherein the eyelid is closed, then a relatively small amount of the composition (and the active agent) will seep through the eyelid margin to reach the conjunctiva. However, in this case, the amount reaching the conjunctiva can sufficiently treat the red eye, and the excess composition on the ocular area that did not reach the conjunctiva does not provide any unintended side effect. Thus, in some embodiments, less than 50%, 40%, 30%, 20%, 10%, 5%, 4%, 3%, 2%, or 1% of the active agent reaches the area to be treated.

In some embodiments, the active agent is an antihistamine. An antihistamine is any composition or compound which serves to reduce or eliminate effects mediated by histamine, an endogenous chemical mediator released during allergic reactions, through action at the histamine receptor. In some embodiments, the antihistamine is selected from the group consisting of antazoline phosphate, pheniramine maleate, and combinations thereof. In some embodiments, the antihistamine can aid in reducing an ocular allergic response, thus treating the symptoms of the ocular allergy. For example, while not being bound by any theory, the boric acid in the composition can aid in removing debris (e.g., allergens) associated with the ocular allergy, and the antihistamine can aid in treating the symptoms of the ocular allergy, e.g., reduce the itching, redness and swelling associated with ocular allergies.

In some embodiments, the active agent is a vasoconstrictor. A vasoconstrictor is a pharmaceutically acceptable agent used to narrow or constrict an opening of a blood vessel. When used ophthalmically, a vasoconstrictor can constrict the blood vessels of the eyelid margin and/or conjunctiva, resulting in a reduction in the amount of redness of the eyelid margin and the whitening of the conjunctiva. Vasoconstrictors are known to those in the art and can be selected from the group consisting of, but not limited to, naphazoline hydrochloride, tetrahydrozoline hydrochloride and oxymetazoline hydrochloride. Various concentrations of vasoconstrictor can be used in the present invention. For example, the vasoconstrictor can be about 0.001% to about 0.1% w/v of the composition, about 0.005% to about 0.05% w/v of the composition, about 0.005% to about 0.02% w/v of the composition, or about 0.01% w/v of the composition. In some embodiments, the vasoconstrictor aids in treatment of blepharitis and red eye. For example, while not being bound by a particular theory, in some embodiments the boric acid of the composition can aid in removing debris associated with causing blepharitis, and the vasoconstrictor can aid in treating the symptoms of blepharitis, whitening the conjunctiva, and removing redness from the eyelid margin.

In some embodiments, the active agent is a mast cell stabilizer. A mast cell stabilizer includes any compound or composition which blocks mast cell degranulation, thereby preventing the release of histamine and related mediators. In some embodiments, the mast cell stabilizer is selected from the group consisting of olopatadine, ketotifen, epinastine, azelastine, pemirolast, nedocromil, and combinations thereof. In some embodiments, the mast cell stabilizer can aid in reducing an ocular allergic response, thus treating the symptoms of the ocular allergy. For example, while not being bound by any theory, the boric acid in the composition can aid in removing debris (e.g., allergens) associated with the ocular allergy, and the mast cell stabilizer can aid in treating the symptoms of the ocular allergy, e.g., reduce the itching, redness and swelling associated with ocular allergies.

In some embodiments, the active agent is a nonsteroidal anti-inflammatory agent. Non-steroidal anti-inflammatory drugs, usually abbreviated to NSAIDs, are compounds or compositions with analgesic, antipyretic and anti-inflammatory effects, wherein the effects are achieved by non-selective inhibition of the enzyme cyclooxygenase, inhibiting the cyclooxygenase-1 (COX-1) and/or cyclooxygenase-2 (COX-2) isoenzymes. In some embodiments, the nonsteroidal anti-inflammatory agent is selected from the group consisting of ketorolac tromethamine, flurbiprofen, diclofenac, nepafenac, and combinations thereof. For example, while not being bound by any theory, the boric acid in the composition can aid in removing debris (e.g., allergens) associated with the ocular allergy, and the NSAID can aid in treating the symptoms of the ocular allergy, e.g., reduce the itching, redness and swelling associated with ocular allergies.

In some embodiments, the composition of the present invention further comprises an artificial tear solution. In some embodiments, the artificial tear solution can comprise water, a demulcent, a tonicity adjuster, and a buffer. The term “artificial tears” refers to any composition suitable moistening and lubricating the conjunctiva and eyelid.

Various demulcents can be used. The term “demulcent” refers to any compound or composition that when applied to an ocular area can lubricate, soothe and/or protect the mucous membrane of the eye. In some embodiments, the demulcent is selected from a cellulose derivative, dextran, gelatin, polyol, polyvinyl alcohol, povidone, chondroitin sulfate, hyaluronic acid, and combinations thereof. Various cellulose derivatives can be used. Examples include, but are not limited to, carboxymethylcellulose, one or more salts of carboxymethylcellulose, hydroxyethyl cellulose, hypromellose, methylcellulose, and combinations thereof. The term “polyol” refers to a compound with greater than 2 alcohol groups. Examples of polyols include, but are not limited to glycerin, polyethylene glycol, polysorbate, propylene glycol, and combinations thereof. Various concentrations of polyols can be used in the present invention. In some embodiments, the polyol is about 0.01% to about 20.0% w/v of the composition, about 0.1% to about 10.0% w/v of the composition, or about 0.5% to about 5.0% w/v of the composition.

While not being bound by any theory, a composition of the present invention that contains artificial tears can be used to treat the symptoms associated with blepharitis, dry eyes, red eyes, and/or allergic conjunctivitis by increasing the lubrication on the conjunctiva. Thus, in some embodiments, the boric acid can aid in removal of debris, and the artificial tears can aid in lubricating the conjunctiva, thereby providing relief from various conditions.

The composition of the present invention can further comprise an ultraviolet (UV) absorbing compound. The term “UV absorbing” refers to any pharmaceutically acceptable compound suitable for placement in the ocular area that absorbs ultraviolet radiation. Various UV absorbing compounds are known to those in the art and can be selected from, but not limited to, vitamin E acetate, vitamin E succinate, and vitamin E polyethylene glycol succinate.

Various concentrations of the UV-absorbing compounds can be used. For example, the UV-absorbing compound can be about 0.1% to about 10% w/v of the composition, about 0.5% to about 7% w/v of the composition, about 1% to about 5% w/v of the composition, about 1.5% to about 3% w/v of the composition, or about 2% w/v of the composition.

In some embodiments, the composition of the present invention further comprises one or more additional ingredients selected from the group consisting of a buffer, tonicity adjuster, viscosity modifier, performance enhancer, and combinations thereof. In some embodiments, these ingredients are selected from sodium chloride, sodium phosphates, potassium phosphates, citric acid, sodium citrate, acetic acid, sodium acetate, glycerin, propylene glycol, polyethylene glycol, and polysorbates. In some embodiments of the present invention, the composition is substantially free of synthetic detergents and cleansers.

Acid, bases, and/or buffers preferably can be included to provide and/or maintain the present compositions at a pH in the physiologically acceptable range, more preferably in a range of about 3 to about 9, or 4 to about 8.5, still more preferably about 5 to about 8.5 or about 5.5 to 8.0, and especially about 6.0 to 8.0 or 6.1 to about 7.8.

The term buffer refers to a pharmaceutically and ophthalmically acceptable compound or composition that is capable of neutralizing both acids and bases and thereby maintaining the original acidity or basicity of the composition. Buffers can include, but are not limited to, phosphate buffers (e.g., sodium and potassium phosphates), phosphates, bicarbonate, citrate, borate, ACES, BES, BICINE, BIS-Tris, BIS-Tris Propane, HEPES, HEPPS, imidazole, MES, MOPS, PIPES, TAPS, TES, Tricine, acetate buffers, citrate buffers, tromethamine and combinations thereof.

Acids optionally useful in the present compositions include boric acid, hydrochloric acid, acetic acid, other acids which are ophthalmically acceptable in the concentrations used, and the like.

Bases which may be included in the present compositions include, but are not limited to, sodium and/or potassium hydroxides, other alkali and/or alkaline earth metal hydroxides, organic bases, other bases which are ophthalmically acceptable in the concentrations used, and the like.

Tonicity adjusters can be used to adjust the salt concentration of the composition, provided they are ophthalmically acceptable. Tonicity adjusters optionally useful in the present compositions include, but are not limited to, dextrose, potassium chloride and/or sodium chloride and the like, preferably sodium chloride. In some embodiments, the tonicity adjuster is used to produce an isotonic composition. In some embodiments, the tonicity adjuster is used to produce a hypotonic composition.

Viscosity modifiers can be used to adjust the coefficient of viscosity for the composition of the present invention. Increasing the coefficient of viscosity can increase the retention time of the composition on the ocular area. Viscosity modifiers optionally useful in the compositions of the present invention include, but are not limited to, carbopol, cellulose derivatives such as hydroxypropylmethyl cellulose, sodium carboxymethyl cellulose, hydroxyethyl cellulose, other viscosity-inducing materials useful in ophthalmic formulations and the like.

In some embodiments of the present invention, the composition further comprises polymers. In some embodiments, the polymers can increase the adhesive power of the towelette to enhance performance of the composition of the present invention. Polymers can be selected from the group consisting of polyvinyl alcohol, povidone, various cellulose derivatives, natural gums, carbomer polymers, and the like. In some embodiments, the polymer is present at about 0.0001% to about 1.0% w/v of the composition, about 0.001% to about 1.0% w/v of the composition, or about 0.005% to about 0.5% w/v of the composition. In some embodiments, the polymer is present at about 0.001% to about 0.01% w/v of the composition.

The present compositions may include effective amounts of chelating or sequestering components other than, or in addition to, ethylene diamine tetracetic acid, such as citric acid, tartaric acid and the like.

As used herein, the term “pharmaceutically acceptable” refers to those compounds, materials, compositions, and/or solutions which are, within the scope of sound medical or veterinary judgment, suitable for contact with the tissues of human beings and animals without excessive toxicity, irritation, allergic response, or other problem complications commensurate with a reasonable benefit/risk ratio. As used herein, the term “ophthalmically acceptable” refers to those compounds, materials, compositions, and/or solutions which are, within the scope of sound medical or veterinary judgment, suitable specifically for contact with the tissues of the eye, and the area surrounding the eye without excessive toxicity, irritation, allergic response, or other problem complications commensurate with a reasonable benefit/risk ratio.

The present compositions can be in any suitable physical form effective to be administered to the eye. Such forms include liquids (either solutions or suspensions), semi-solids (gels, creams, ointments, etc.), solids and the like. Each of these physical forms of the present compositions can be prepared using techniques and processing which are conventional and well known in the art. For a more detailed discussion of the preparation and administration of ophthalmic formulations see Remington's Pharmaceutical Sciences, 15 ed., pgs. 1489 to 1504 (1975) which is incorporated in its entirety herein by reference.

In the present invention, the composition can be “applied” to an ocular area. In some embodiments, the term “applied” or “applying” refers to the placement of the composition directly on the ocular surface. In some embodiments, the term applying further comprises agitating the composition on the surface, for example with a finger or other bodily appendage, with a towelette, or other object suitable for spreading the composition and/or agitating the surface to facilitate removal of the debris. In some embodiments, the term “applied” or “applying” refers to the indirect administration of the composition to an ocular surface. For example, in some embodiments the term “applied” or “applying” refers to the placement of the composition in a towelette (or other suitable object) to form a wetted towelette, and then placing the wetted towelette on the ocular area. In some embodiments, the term “applied” or “applying” further comprises agitating the ocular area to aid in removing the debris. In some embodiments, the eyelid of the subject being treated is closed when the composition is applied. In some embodiments in which the eyelids are closed, the composition of the present invention enters between the eyelids and comes in contact with the conjunctiva and eyelid margins.

In the present invention, the term ocular area refers to all or part of the external skin surrounding the eye, i.e., the eyelid and the margin of the eyelid, and associated hair projecting therefrom, i.e., eyelashes and eye brows. The ocular area can be present on any animal having an eyelid. Thus, methods of the present invention are applicable to both human use and veterinary use, preferably for human use or for use on dogs and/or cats.

In some embodiments of the present invention, the composition of the present invention can be used to remove debris from bodily surfaces other than an ocular area. In some embodiments, the composition of the present invention can be used to remove debris from the face, e.g., the nose, cheek, lips, chin, forehead, etc. In some embodiments, the composition of the present invention can be used to remove debris from any skin covered surface, e.g., neck, chest, hands, arms, legs, feet, etc.

The concentration of the boric acid can vary, provided that it is administered in an ophthalmically acceptable dosage concentration. In some embodiments, the boric acid is about 0.1% to about 5% w/v of the composition, about 0.5% to about 3% w/v of the composition, or about 1% to about 2% w/v of the composition. One of skill in the art will understand that in solution, boric acid in the presence of a salt can form a borate salt (e.g., sodium borate). One of skill in the art can vary the boric acid/sodium borate ratio to achieve the desired pH and tonicity levels.

The present invention is directed to methods of removing debris utilizing a composition comprising the preservatives benzalkonium chloride and edetate disodium. Examples of alternative ophthalmic preservatives are well known to those in the art and include, but are not limited to, sodium perborate, polyquaternium-1, cetylpyridinium chloride, chlorobutanol, methylparaben, sodium benzoate, and sorbic acid. In some embodiments, the edetate disodium is present in a concentration of about 0.001% to about 1% w/v, preferably about 0.01% to about 0.4% w/v, or most preferably about 0.2% w/v. In some embodiments, the benzalkonium chloride is about 0.0005% to about 0.5% w/v, 0.001% to about 0.1% w/v, about 0.005% to about 0.04% w/v, about 0.005% to about 0.03% w/v, or most preferably about 0.01% to about 0.03% w/v.

In some embodiments, the present invention is directed to a method for treating one of more conditions selected from blepharitis, dry eyes, red eyes, and allergic conjunctivitis, the method comprising: (a) applying a composition to an ocular area containing debris, the composition comprising (i) about 0.1% to about 5.0% w/v boric acid; (ii) about 0.001% to about 1.0% w/v edetate disodium; and (iii) about 0.001% to about 1.0% w/v benzalkonium chloride; and (b) removing the debris from the ocular area In some embodiments when treating the condition, the composition further comprises a vasoconstrictor, antihistamine, mast cell stabilizer, and/or nonsteroidal anti-inflammatory. In some embodiments, the applying is performed by contacting a towelette comprising the composition to the ocular area.

Blepharitis, as used herein, refers to long-lasting or chronic inflammation of the eyelids, particularly at the lid margins. Symptoms associated with blepharitis include general eye discomfort, redness of the eye, excessive tearing, burning, stinging, foreign body sensation, itching, light sensitivity (photophobia), and an irritating, sandy, gritty sensation that is often worse upon awakening, red and swollen eyelids, blurred vision, frothy tears, and crusting of the eyelashes upon awakening. Severe and/or chronic effects of blepharitis can include thickened lid margins, dilated and visible capillaries, trichiasis, eyelash loss, ectropion and entropion. As used herein, treatment of blepharitis by the composition described herein can treat one, or more than one (e.g., two, three, four, or five), of the above listed symptoms. Blepharitis can sometimes be classified as two different forms: anterior blepharitis and posterior blepharitis. Anterior blepharitis affects the outer margin of the eyelids, where the eyelashes are located. Posterior blepharitis affects the inner eyelid where the meibomian oil glands are located. Blepharitis may be anterior, posterior, or a combination of anterior and posterior. The method of the present invention can be used to treat one or both forms of blepharitis

The term “dry eyes,” also known as Keratitis sicca, refers to a condition wherein not enough tears are present on the surface of the eye. The lack of tears on the surface of the eye can be the result of insufficient tear production, or too much drainage of tears that would normally be sufficient. Symptoms associated with dry eyes range from mild irritation and a sensation of something in the eye, to severe discomfort and sensitivity to light.

The term “red eyes” refers to a condition wherein enlarged, dilated blood vessels lead to the appearance of redness on the surface of the eye.

The term “ocular allergy” refers to a sensitivity to allergens that, under normal conditions, are considered innocuous to others. The terms “ocular allergy” includes allergic conjunctivitis and allergic rhinoconjuctivitis. An ocular allergy includes inherited allergies as well as seasonal and perennial allergies. Symptoms include itching, redness, swelling, burning sensation, watery eyes (excess tearing), or sometimes mild mucoid discharge from the eye. An ocular allergy can affect one or both eyes of the subject being affected.

The terms “treating”, “treatment” or “treats” refer to the administering to a subject a composition of the present invention for purposes which can include prevention, amelioration, or cure of blepharitis, or the symptoms thereof.

In some embodiments, the present invention is directed to a pharmaceutical kit for removing debris from the ocular area, the kit comprising (a) a composition comprising (i) about 0.1% to about 5.0% w/v boric acid; (ii) about 0.001% to about 1.0% w/v edetate disodium; and (iii) about 0.001% to about 1.0% w/v benzalkonium chloride; and (b) one or more towelettes.

The towelettes can be individually packaged, e.g., in polyethylene-lined foil pouches. Alternatively, the towelettes can be packaged as multiple units, e.g., in resealable glass or plastic packages. In some pharmaceutical kits, the composition is added to the towelette immediately preceding the application to the ocular area. Therefore, in some embodiments the kit comprises non-wetted towelettes and the composition provided in a separate container, e.g., a plastic bottle. Optionally, the pharmaceutical kit of the present invention can include a dropper or other device for transferring the composition to a towelette.

Optionally, the kit can further comprise printed matter containing instructions for using the wetted towelettes to remove debris. For example, such printed instructions can be in a form prescribed by a governmental agency regulating the manufacture, use or sale of pharmaceuticals or biological products, which notice reflects approval by the agency of the manufacture, use or sale for human application. In some embodiments, the kit further comprises printed matter, which, e.g., provides information on the use of the pharmaceutical composition or a pre-recorded media device which, e.g., provides information on the use of the method of the present invention.

“Printed matter” can be, for example, a book, booklet, brochure, leaflet or the like. The printed matter can describe the use of the pharmaceutical composition of the present invention. Possible formats included, but are not limited to, a bullet point list, a list of frequently asked questions (FAQ) or a chart. Additionally, the information to be imparted can be illustrated in non-textual terms using pictures, graphics or other symbols.

The kit can also include a container for storing the components of the kit. The container can be, for example, a bag, box, envelope or any other container that would be suitable for use in the present invention. In some embodiments, the container is large enough to accommodate each component of the present invention. However, in some cases, it can be desirable to have a smaller container which is large enough to carry only some of the components of the present invention.

### EXAMPLES

### Example 1

Two different formulations of the present invention are described in Table 1.

TABLE 1

| Ingredient | Formulation 1 | Formulation 2 |
|---|---|---|
| Boric Acid | 1% w/v | 1.5% w/v |
| Edetate Disodium | 0.2% w/v | 0.2% w/v |
| Benzalkonium Chloride | 0.01% w/v | 0.02% w/v |
| Phosphate Buffer | 0.005 (molar) | 0.005 molar |
| Sodium Chloride | 0.4% w/v | — |
| Sodium Borate Decahydrate | — | 0.6% w/v |
| Sodium Hydroxide/HCl | add to achieve | add to achieve |
| | pH 7.8 | pH 7.9 |
| Purified Water | q.s. 100 mL | q.s. 100 mL |

### Example 2

A formulation of the present invention containing a vasoconstrictor (Formulation 3) is described in Table 2.

TABLE 2

| Ingredient | Formulation 3 |
|---|---|
| Boric Acid | 1.5% w/v |
| Edetate Disodium | 0.2% w/v |
| Benzalkonium Chloride | 0.02% w/v |
| Phosphate Buffer | 0.005 molar |
| Sodium Chloride | 0.007% w/v |
| Sodium Borate Decahydrate | 0.6% w/v |
| Sodium Hydroxide/HCl | add to achieve pH 6.1 |
| Tetrahydrozoline Hydrochloride | 0.01% w/v |
| Purified Water | q.s. 100 mL |

### Example 3

A formulation of the present invention containing an absorber of UV radiation (Formulation 3) is described in Table 3.

TABLE 3

| Ingredient | Formulation 4 |
|---|---|
| Boric Acid | 1.5% w/v |
| Edetate Disodium | 0.2% w/v |
| Benzalkonium Chloride | 0.02% w/v |
| Sodium Borate Decahydrate | 0.6% w/v |
| Vitamin E Acetate | 0.2% w/v |
| Vitamin E Polyethylene Glycol | 2% w/v |
| Succinate | |
| Sodium Hydroxide/HCl | add to achieve pH 7.0 |
| Purified Water | q.s. 100 mL |

It is to be appreciated that the Detailed Description section, and not the Summary and Abstract sections, is intended to be used to interpret the claims. These examples illustrate possible compositions used in the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A composition for removing debris from an ocular area comprising:
(a) about 0.1% to about 5.0% w/v boric acid;
(b) about 0.001% to about 1.0% w/v edetate disodium; and
(c) about 0.001% to about 1.0% w/v benzalkonium chloride.

2. The composition of claim 1, wherein the removed debris is a cosmetic product.

3. The composition of claim 1, wherein the removed debris is a biological product resulting from an eye infection or eye-inflammation.

4. The composition of claim 3, wherein the biological product is encrusted mucin or dead tissue scales.

5. The composition of claim 1, wherein the composition further comprises a active agent.

6. The composition of claim 5, wherein the active agent is selected from the group consisting of an antihistamine, vasoconstrictor, mast cell stabilizer, nonsteroidal anti-inflammatory agent, and combinations thereof.

7. The composition of claim 6, wherein the active agent is an antihistamine.

8. The composition of claim 7, wherein the antihistamine is selected from the group consisting of antazoline phosphate, pheniramine maleate, and combinations thereof.

9. The composition of claim 6, wherein the active agent is a vasoconstrictor.

10. The composition of claim 9, wherein the vasoconstrictor is selected from naphazoline hydrochloride, tetrahydrozoline hydrochloride or oxymetazoline hydrochloride.

11. The composition of claim 6, wherein the vasoconstrictor is about 0.001% to about 0.1% w/v of the composition.

12. The composition of claim 6, wherein the active agent is a mast cell stabilizer.

13. The composition of claim 12, wherein the mast cell stabilizer is selected from the group consisting of olopatadine, ketotifen, epinastine, azelastine, pemirolast, nedocromil, and combinations thereof.

14. The composition of claim 6, wherein the active agent is a nonsteroidal anti-inflammatory agent.

15. The composition of claim 14, wherein the nonsteroidal anti-inflammatory agent is selected from the group consisting of ketorolac tromethamine, flurbiprofen, diclofenac, nepafenac, and combinations thereof.

16. The composition of claim 1 further comprising an artificial tear solution.

17. The composition of claim 16, wherein said artificial tear solution comprises water, a cellulose derivative, and an antimicrobial.

18. The composition of claim 1, wherein the composition further comprises a UV-absorbing compound.

19. The composition of claim 18, wherein the UV-absorbing compound is selected from vitamin E acetate, vitamin E succinate, or vitamin E polyethylene glycol succinate.

20. The composition of claim 19, wherein the UV-absorbing compound is about 0.1% to about 10% w/v of the composition.

21. The composition of claim 1, wherein the composition further comprises an additional ingredient selected from the group consisting of a buffer, tonicity adjuster, viscosity modifier, performance enhancer, and combinations thereof.

22. The composition of claim 21, wherein the ingredient is selected from sodium chloride, sodium phosphates, potassium phosphates, citric acid, sodium citrate, acetic acid, sodium acetate, glycerin, propylene glycol, polyethylene glycol, and polysorbates.

23. The composition of claim 1, wherein the ocular area comprises the eyelid, the margin of the eyelid, the eyelashes, the eye brow, or combinations thereof.

24. The composition of claim 1 comprising about 0.5% to about 3% w/v boric acid.

25. The composition of claim 1 comprising about 0.01% to about 0.4% w/v edetate disodium.

26. The composition of claim 1 comprising about 0.005% to about 0.04% w/v benzalkonium chloride.

27. A composition for removing debris from an ocular area comprising:
(a) 1% w/v boric acid;
(b) 0.2% w/v edetate disodium;
(c) 0.01% w/v benzalkonium chloride;
(d) 0.005 molar phosphate buffer;
(e) 0.4% w/v sodium chloride;
(f) sodium hydroxide/HCl sufficient to adjust the pH to 7.8; and
(g) purified water.

28. A composition for removing debris from an ocular area comprising:
(a) 1.5% w/v boric acid;
(b) 0.2% w/v edetate disodium; and
(c) 0.02% w/v benzalkonium chloride;
(d) 0.005 molar phosphate buffer;
(e) 0.007% w/v sodium chloride;
(f) 0.6% w/v sodium borate decahydrate;
(g) 0.01% w/v tetrahydrozoline hydrochloride;
(h) sodium hydroxide/HCl sufficient to adjust the pH to 7.8; and
(i) purified water.

29. A composition for removing debris from an ocular area comprising:
(a) 1.5% w/v boric acid;
(b) 0.2% w/v edetate disodium; and
(c) 0.02% w/v benzalkoniurn chloride;
(d) 0.02% w/v vitamin E acetate;
(e) 2% w/v vitamin E polyethylene glycol succinate;
(f) 0.6% w/v sodium borate decahydrate;
(g) sodium hydroxide/HCl sufficient to adjust the pH to 7.8; and
(h) purified water.

30. A method for removing debris from an ocular area, the method comprising applying a wetted towelette to an ocular area to remove debris, the wetted towelette comprising a composition comprising:
(i) about 0.1% to about 5.0% w/v boric acid;
(ii) about 0.001% to about 1.0% w/v edetate disodium; and
(iii) about 0.001% to about 1.0% w/v benzalkonium chloride.

31. The method of claim 30, wherein the towelette is wetted immediately before the application to the ocular area.

32. The method of claim 30, wherein the towelette is wetted substantially before the application to the ocular area.

33. The method of claim 30, wherein the towelette is disposable.

34. The method of claim 30, wherein the removed debris is a cosmetic product.

35. The method of claim 34, wherein the cosmetic product is selected from the group consisting of mascara, eye shadow makeup, eyeliner, moisturizer, makeup base, concealer, eyebrow pencil, glitter and combinations thereof.

36. The method of claim 30, wherein the removed debris is a biological product resulting from an eye infection or eye inflammation.

37. The method of claim 36, wherein the biological product is encrusted mucin or dead tissue scales.

38. The method of claim 30, the composition further comprising a vasoconstrictor.

39. The method of claim 38, wherein the vasoconstrictor is selected from naphazoline hydrochloride, tetrahydrozoline hydrochloride or oxymetazoline hydrochloride.

40. The method of claim 38, wherein the vasoconstrictor is about 0.001% to about 0.1% w/v of the composition.

41. The method of claim 30, the composition further comprising a UV-absorbing compound.

42. The method of claim 41, wherein the UV-absorbing compound is selected from vitamin E acetate, vitamin E succinate, or vitamin E polyethylene glycol.

43. The method of claim 42, wherein the UV-absorbing compound is about 0.1% to about 10% w/v of the composition.

44. The method of claim 30, the composition further comprising an additional ingredient selected from the group consisting of a buffer, tonicity adjuster, viscosity modifier, performance enhancer, and combinations thereof.

45. The method of claim 44, wherein the ingredient is selected from sodium chloride, sodium phosphates, potassium phosphates, citric acid, sodium citrate, acetic acid, sodium acetate, glycerin, propylene glycol, polyethylene glycol, and polysorbates.

46. The method of claim 30, wherein the ocular area comprises the eyelid, the margin of the eyelid, the eyelashes, the eye brow, or combinations thereof.

47. The method of claim 30 comprising about 0.5% to about 3% w/v boric acid.

48. The method of claim 30 comprising about 0.01% to about 0.4% w/v edetate disodium.

49. The method of claim 30 comprising about 0.005% to about 0.04% w/v benzalkonium chloride.

50. A method for removing a cosmetic product from an ocular area, the method comprising applying a wetted towelette to an ocular area to remove the cosmetic product, the wetted towelette comprising a composition comprising:
(i) about 0.1% to about 5.0% w/v boric acid;
(ii) about 0.001% to about 1.0% w/v edetate disodium; and
(iii) about 0.001% to about 1.0% w/v benzalkonium chloride.

51. The method of claim 50, wherein the cosmetic product is selected from group consisting of mascara, eye shadow makeup, eyeliner, moisturizer, makeup base, concealer, eyebrow pencil, glitter and combinations thereof.

52. A method for treating one of more conditions selected from blepharitis, dry eyes, red eyes, and allergic conjunctivitis, the method comprising:
(a) applying a composition to an ocular area containing debris, the composition comprising:
(i) about 0.1% to about 5.0% w/v boric acid;
(ii) about 0.001% to about 1.0% w/v edetate disodium; and
(iii) about 0.001% to about 1.0% w/v benzalkonium chloride; and
(b) removing the debris from the ocular area.

53. The method of claim 52, the composition further comprises a vasoconstrictor.

54. The method of claim 52, the composition further comprising an antihistamine.

55. The method of claim 52, the composition further comprising a mast cell stabilizer.

56. The method of claim 55, the composition further comprising a nonsteroidal anti-inflammatory agent.

57. The method of any one of claims 52-56, wherein the applying is performed by contacting a towelette comprising the composition to the ocular area.

58. A kit for removing debris from the ocular area, the kit comprising
(a) a composition comprising:
(i) about 0.1% to about 5.0% w/v boric acid;
(ii) about 0.001% to about 1.0% w/v edetate disodium; and
(iii) about 0.001% to about 1.0% w/v benzalkonium chloride; and
(b) one or more towelettes.

59. The kit of claim 58, further comprising instructions for using the composition of (a) and the towelette of (b) to remove debris.
